(19) United States
(12) Patent Application Publication
(10) Pub. No.: US 2021/0315533 A1
(43) Pub. Date: Oct. 14, 2021

(54) MAMMOGRAPHY APPARATUS AND MAMMOGRAPHY METHOD

(71) Applicant: VIEWORKS CO., LTD., Anyang-si (KR)

(72) Inventor: Seung Gyu KIM, Anyang-si (KR)

(73) Assignee: VIEWORKS CO., LTD., Anyang-si (KR)

(21) Appl. No.: 17/226,221

(22) Filed: Apr. 9, 2021

(30) Foreign Application Priority Data
Apr. 10, 2020 (KR) 10-2020-0044218

(51) Int. Cl. A61B 6/00; A61B 6/04

(52) U.S. Cl. CPC A61B 6/502; A61B 6/44; A61B 2562/0247; A61B 6/0435; A61B 6/469; A61B 6/0414

(57) ABSTRACT

The present invention relates to a mammography apparatus and a mammography method. The present invention provides a mammography apparatus and a mammography method including: a preliminary imaging step of performing imaging on a breast by emitting radiation in accordance with a first imaging condition; a second imaging condition determination step of determining a second imaging condition in accordance with a first image acquired in the preliminary imaging step; and a main imaging step of performing imaging on the breast by emitting the radiation on the basis of the second imaging condition, in which a uncompressed region in which the breast is not compressed is excluded when determining the second imaging condition.

### CROSS-REFERENCE TO RELATED APPLICATIONS

This application claims priority to and the benefit of Korean Patent Application No. 10-2020-0044218 filed in the Korean Intellectual Property Office on Apr. 10, 2020, the entire contents of which are incorporated herein by reference.

### TECHNICAL FIELD

The present invention relates to a mammography apparatus and a mammography method. More particularly, the present invention relates to a mammography apparatus and a mammography method which optimize radiographic imaging conditions such as radiation exposure conditions.

### BACKGROUND ART

In general, mammography, which refers to a radiographic breast imaging technology for capturing images of a breast by using radiation such as X-rays, has various advantages of radiography and has a unique feature that may minimize the exposure by enlarging images, reducing the number of image capturing processes, increasing the resolution, and adjusting the brightness and contrast ratio, as a result of which the use of mammography is rapidly growing.

The mammography apparatus may include a column disposed perpendicular to a floor and having a columnar shape, a C-arm having a middle part connected to the column so as to be movable upward and downward and rotatable along the column, the C-arm having a C shape or a shape similar thereto as a whole by being bent in an arc shape so that two opposite ends thereof face each other, a radiation generator mounted at one end of the C-arm and configured to emit radiation to the other end of the C-arm that faces one end, a detector disposed to face the radiation generator, and a compressing unit (also referred to as a ‘compression paddle’) for compressing a breast.

During the mammography using the radiation such as X-ray, it is desirable to minimize radiation dose to the breast. As a method of controlling the radiation exposure, automatic exposure control (AEC) may be applied, which adopts a dose detecting sensor for detecting the dose of radiation passing through the breast and controls and stops the emission of the radiation when a required dose of radiation is emitted. Also, the radiographic imaging process is divided into preliminary imaging (a pre-exposure) and main imaging, and an imaging condition for the main imaging is determined on the basis of a radiographic image acquired by irradiating the breast with a low dose of radiation in the preliminary imaging.

When performing the mammography using the radiation, the preliminary imaging and the main imaging are performed in a state in which the breast is compressed by the compressing unit. However, a degree to which the breast is compressed by the compressing unit may vary depending on respective portions of the breast. However, in the related art, because a main imaging condition is set without considering the degree to which the breast is compressed by the compressing unit, there is a problem in that imaging quality is not uniform, an image of a partial region of the breast is not clear, or excessive radiation exposure occurs.

### RELATED ART DOCUMENT

### Patent Document

Japanese Patent Application Laid-Open No. 2013-103002

### SUMMARY OF THE INVENTION

The present invention has been made in an effort to provide a mammography apparatus and a mammography method which set a radiographic imaging condition in consideration of a state of a breast compressed by a compressing unit and acquire a radiographic image of the breast.

The present invention provides a mammography method including: a preliminary imaging step of performing imaging on a breast by emitting radiation in accordance with a first imaging condition; a second imaging condition determination step of determining a second imaging condition in accordance with a first image acquired in the preliminary imaging step; and a main imaging step of performing imaging on the breast by emitting the radiation on the basis of the second imaging condition, in which a uncompressed region in which the breast is not compressed by a compressing unit is excluded when determining the second imaging condition.

In the exemplary embodiment, in the second imaging condition determination step, the uncompressed region may be determined on the basis of a breast thickness t by which the breast is compressed by the compressing unit.

A length of the uncompressed region from a tip of the breast may be smaller than the breast thickness t. For example, a length of the uncompressed region may be set by multiplying the breast thickness t by a positive (+) value smaller than 1.

The uncompressed region may be determined by modeling a front shape of the breast as any one of a hemispheric shape, an elliptical shape, and a parabolic shape.

In the exemplary embodiment, in the second imaging condition determination step, the uncompressed region may be determined in consideration of a change in pixel value in the first image.

In the exemplary embodiment, in the second imaging condition determination step, the uncompressed region may be determined by semantic segmentation on the first image.

In the exemplary embodiment, the uncompressed region may be determined by using a sensing value of a pressure sensor unit provided on an upper surface of a detection unit or a tray for supporting the breast or a lower surface of the compressing unit for compressing the breast.

The second imaging condition determination step may further include a step of excluding a pectoralis region or an implant region from the first image.

The second imaging condition determination step may further include a step of setting a mammary gland region to a region of interest in the first image, and determining the second imaging condition in consideration of the mammary gland region and the breast thickness t.

The present invention further provides a mammography apparatus including: an irradiation unit configured to emit radiation to a breast in accordance with a first imaging condition or a second imaging condition; a compressing unit configured to compress the breast; a detection unit configured to detect the radiation emitted to the breast; and a control unit configured to determine the second imaging condition based on a first image of the breast acquired in accordance with the first imaging condition, and control the irradiation unit and the detection unit, in which the control unit excludes a uncompressed region in which the breast is not compressed, when determining the second imaging condition.

In the exemplary embodiment, the control unit may determine the uncompressed region on the basis of a breast thickness t by which the breast is compressed by the compressing unit.

The control unit may determine the uncompressed region by modeling a front shape of the breast as any one of a hemispheric shape, an elliptical shape, and a parabolic shape.

The control unit may determine the uncompressed region in consideration of a change in pixel value in the first image.

In the exemplary embodiment, the control unit may determine the uncompressed region by semantic segmentation on the first image.

In the exemplary embodiment, the breast may be supported on an upper surface of the detection unit or a tray, a pressure sensor unit may be provided on the upper surface of the detection unit or the tray or a lower surface of the compressing unit, and the control unit may determine the uncompressed region by using a sensing value of the pressure sensor unit.

The control unit may set a region of interest based on a mammary gland region by excluding a pectoralis region or an implant region from the first image.

According to the present invention, it is possible to set the imaging condition for the main imaging of the breast by dividing the image acquired from the preliminary imaging of the breast into the region compressed by the compressing unit and the region not compressed by the compressing unit and extracting the region of interest from the compressed region.

Therefore, there is an advantage of setting an optimum imaging condition and preventing excessive dose of radiation or acquisition of unclear images during the main imaging of the breast. Accordingly, it is possible to reduce the dose of radiation to a patient.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a perspective view illustrating a mammography apparatus according to an exemplary embodiment of the present invention.

FIG. 2 is a view illustrating a state in which a breast is positioned between a compressing unit and a detection unit of the mammography apparatus according to the exemplary embodiment of the present invention.

FIG. 3 is a view illustrating radiation transmittance in the state in which the breast is positioned between the compressing unit and the detection unit of the mammography apparatus as illustrated in FIG. 2.

FIG. 4 is a view exemplarily illustrating a captured breast image.

FIG. 5 is a view illustrating an example in which a control unit of the mammography apparatus according to the exemplary embodiment of the present invention divides a breast into a compressed region and a uncompressed region.

FIG. 6 is a view illustrating a state in which a pressure sensor unit is further provided in the mammography apparatus according to the exemplary embodiment of the present invention and a breast is positioned between the compressing unit and the detection unit.

FIG. 7 is a flowchart illustrating a mammography method according to the exemplary embodiment of the present invention.

FIG. 8 is a flowchart illustrating a process of extracting a region of interest from an image acquired by preliminary imaging in the mammography method according to the exemplary embodiment of the present invention.

FIGS. 9A to 9E are views exemplarily illustrating a process of extracting a region of interest from an image acquired by preliminary imaging in the mammography method according to the exemplary embodiment of the present invention.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter, exemplary embodiments of the present invention will be described in detail with reference to the accompanying drawings. First, in assigning reference numerals to constituent elements of the respective drawings, it should be noted that the same constituent elements will be designated by the same reference numerals, if possible, even though the constituent elements are illustrated in different drawings. In addition, in the description of the present invention, the specific descriptions of publicly-known related configurations or functions will be omitted when it is determined that the specific descriptions may obscure the subject matter of the present invention. Further, the exemplary embodiments of the present invention will be described below, but the technical spirit of the present invention is not limited thereto and may of course be modified and variously carried out by those skilled in the art.

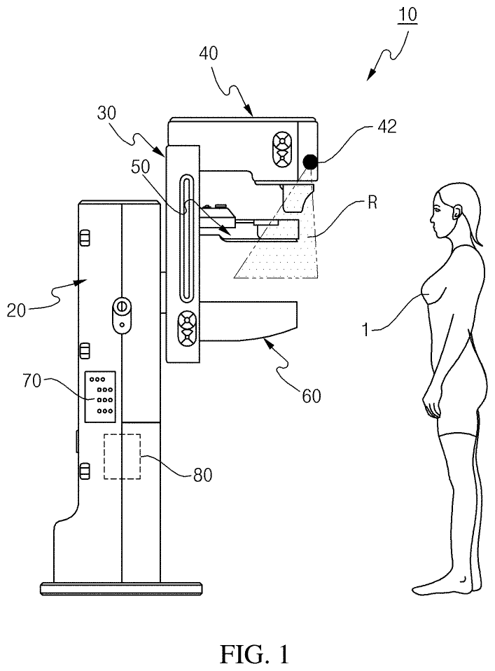

FIG. 1 is a perspective view illustrating a mammography apparatus according to an exemplary embodiment of the present invention.

A mammography apparatus 10 according to an exemplary embodiment of the present invention includes a main body unit 20, a measurement arm unit 30 coupled to the main body unit 20, an irradiation unit 40 provided at an upper end of the measurement arm unit 30 and configured to emit radiation, a compressing unit 50 configured to compress a breast during radiography, a detection unit 60 configured to acquire radiographic images, an input/output unit 70 configured to display information on an input or state of a control instruction, and a control unit 80 configured to control the mammography apparatus 10.

The main body unit 20 may be disposed to be perpendicular to the ground surface and may have a columnar shape.

The measurement arm unit 30 may be coupled to the main body unit 20, and the measurement arm unit 30 may be provided to be rotatable with respect to the main body unit 20.

The irradiation unit 40 has a radiation source 42, and radiation R generated by the radiation source 42 are emitted to a region for radiography.

The compressing unit 50 compresses a breast 1, which is a test subject, for mammography. The compressing unit 50 may be provided on the measurement arm unit 30 so as to be movable upward and downward. The compressing unit 50 may be manually moved upward or downward or moved upward or downward by a motor. In addition, a force or a compressing degree applied to the breast 1 by the compressing unit 50 may be calculated on the basis of current or voltage applied to a separate sensor or the motor. In addition, a lifting height of the compressing unit 50 may be calculated, and a thickness of the breast 1 compressed by the compressing unit 50 may be calculated.

The detection unit 60 detects an image created as the breast 1 is irradiated with the radiation R generated by the irradiation unit 40. The detection unit 60 may be a digital radiation detector. The detection unit 60 may include a scintillator panel that reacts with the radiation, and the detection unit 60 may detect the radiation and convert the radiation into an electrical signal.

The input/output unit 70 may display information on an input or state of a control instruction for operating the mammography apparatus 10. The input/output unit 70 may be configured in the form of a touch panel.

The control unit 80 controls an operation of the mammography apparatus 1. In the exemplary embodiment of the present invention, the control unit 80 may control preliminary imaging and main imaging. Specifically, the control unit 80 may set an imaging condition for the main imaging by using a result of the preliminary imaging of the breast. FIG. 1 illustrates the control unit 80 in the form of a block. The control unit 80 may include hardware including a CPU and software for operating the hardware, and the control unit 80 may be embedded in the main body unit 20.

The present invention is characterized in that when the control unit 80 sets the imaging condition for the main imaging by using the image acquired by the preliminary imaging, the control unit 80 divides the breast image into a compressed region of the breast 1 which is compressed by the compressing unit 50 and a uncompressed region of the breast 1 which is not compressed by the compressing unit 50.

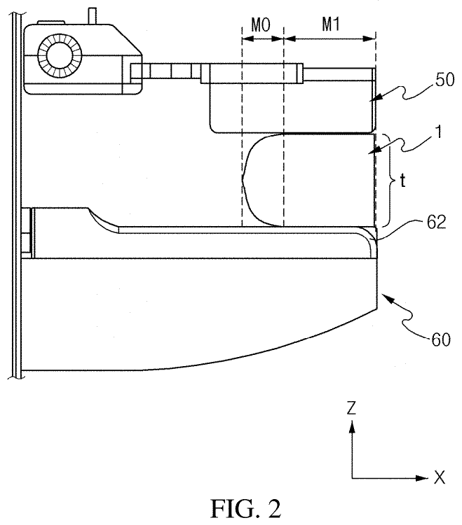

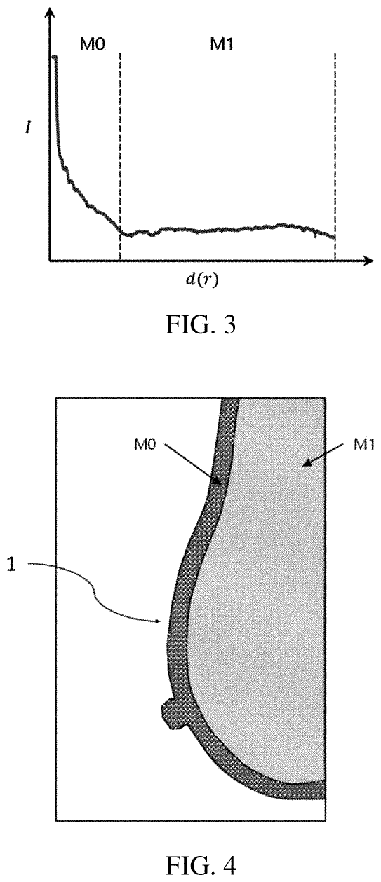

FIG. 2 is a view illustrating a state in which the breast is positioned between the compressing unit and the detection unit of the mammography apparatus according to the present invention, FIG. 3 is a view illustrating radiation transmittance in the state in which the breast is positioned between the compressing unit and the detection unit of the mammography apparatus as illustrated in FIG. 2, and FIG. 4 is a view exemplarily illustrating a captured breast image.

Referring to FIG. 2, when performing imaging on the breast 1, the breast 1 is compressed by the compressing unit 50 in a state in which the breast 1 is positioned on an upper surface of the detection unit 60. In the exemplary embodiment, a tray 62 on which the breast 1 is to be placed may be coupled to the detection unit 60 or provided separately from the detection unit 60.

As illustrated in FIG. 2, when the compressing unit 50 compresses the breast 1 for the mammography, there are the compressed region M1 which is compressed by the compressing unit 50, and the uncompressed region M0 which is not compressed by the compressing unit 50, on the basis of the shape of the breast 1. A thickness of the uncompressed breast 1 is smaller than a thickness t of the breast 1 in the compressed region M1. In the exemplary embodiment, the breast thickness t in the compressed region M1 may be calculated on the basis of a position of the compressing unit 50 in the measurement arm unit 30.

Because a substantial thickness is smaller in the uncompressed region M0 than in the compressed region M1, the radiation transmittance in the uncompressed region M0 is higher than the radiation transmittance in the compressed region M1. In FIG. 3, a horizontal axis represents a distance d(r) from a left side of the breast 1 illustrated in FIG. 2 (a distance in the X-axis direction from a left tip of the breast 1 illustrated in FIG. 2), and a vertical axis represents pixel values I in accordance with the radiation transmittance. Referring to FIG. 3, it can be ascertained that the pixel value is larger in the uncompressed region M0 than in the compressed region M1. In addition, referring to FIG. 4 (FIG. 4 illustrates an example of an MLO (medio lateral oblique) image of the breast 1), the pixel value of the image in the uncompressed region M0 is higher than the pixel value of the image in the compressed region M1.

In a case in which the condition for the main imaging is set based on the uncompressed region M0 from the image acquired by the preliminary imaging, the main imaging is performed with a low dose, and as a result, there is a likelihood that an unclear image may be acquired in the compressed region M1.

Therefore, the present invention is characterized in that when setting the imaging condition for the main imaging by using the image acquired by the preliminary imaging, the control unit 80 sets the imaging condition on the basis of the image in the compressed region M1 except for the image in the uncompressed region M0 among the images acquired by the preliminary imaging.

A method of dividing the breast into the uncompressed region M0 and the compressed region M1 will be described.

In the exemplary embodiment, the control unit 80 may divide the breast image acquired by the preliminary imaging into the uncompressed region M0 and the compressed region M1 by using the pixel value positioned in an inward direction from an outer surface of the breast 1 (also referred to as a ‘skin line’ of the breast). For example, as illustrated in FIG. 4, the breast may be divided into the uncompressed region M0 and the compressed region M1 on the basis of a boundary at which the pixel value is changed. In the exemplary embodiment, an average of the pixel values is obtained by grouping a number of pixels into one group from the outer interface of the breast 1, and an average of the values is obtained by moving the pixel group to the inside of the breast 1, and as a result, it is possible to divide the breast into the uncompressed region M0 and the compressed region M1 on the basis of the point at which an average value of the pixel values is changed. In the exemplary embodiment, the pixel group may be divided into several blocks such as four or nine blocks.

In another exemplary embodiment, the control unit 80 may divide the breast into the uncompressed region M0 and the compressed region M1 by using the breast thickness tin the compressed region M1.

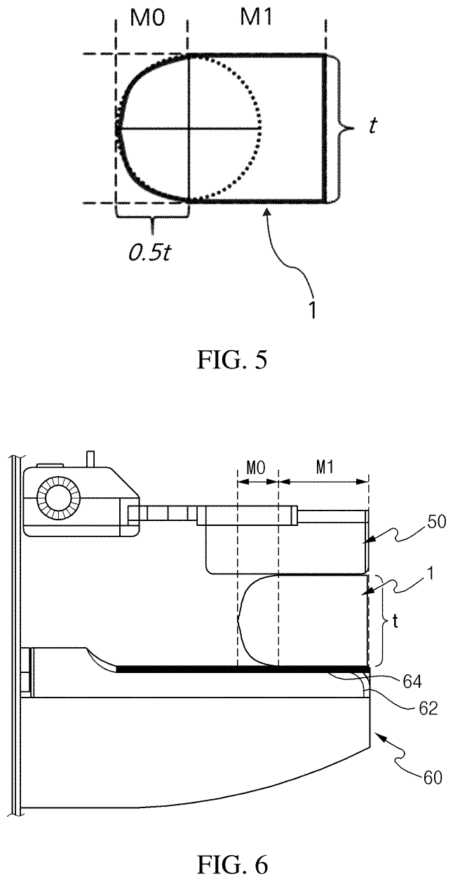

FIG. 5 is a view illustrating an example in which the control unit of the mammography apparatus according to the exemplary embodiment of the present invention divides the breast into the compressed region and the uncompressed region.

Referring to FIG. 5, on the assumption that the uncompressed region M0 of the breast 1 has a hemispheric shape, the breast thickness tin the compressed region M1 may be a diameter of the hemispheric shape, and a horizontal distance of the uncompressed region M0 may be set to be 0.5 t. However, FIG. 5 is only an example, and the shape of the uncompressed region M0 of the breast 1 may be modeled and applied as a parabolic or elliptical shape. Based on the modeling result, a distance of the uncompressed region M0 from the outer interface of the breast 1 may be less or more than 0.5 t. In addition, FIG. 5 illustrates the uncompressed region M0 in a direction in which the breast is away from the chest muscles, but an outer circumferential portion of the breast may be included in the uncompressed region M0. In addition, it may be possible to model and apply a CC (cranial-caudal) view and an MLO view of the breast in different ways.

It is possible to divide the breast into the uncompressed region M0 and the compressed region M1 in consideration of the model illustrated in FIG. 5 as well as a relationship between the distance d(r) from the outer interface of the breast 1 illustrated in FIG. 3 and the pixel value I made in accordance with the dose of the radiation. In this case, the uncompressed region M0 may be set within a range of d(r)<0.5 t. In other words, within a distance made by multiplying the breast thickness t in the compressed region M1 by a predetermined value, the breast may be divided into the uncompressed region M0 and the compressed region M1 on the basis of the boundary at the point at which the pixel value I is greatly changed.

In still another exemplary embodiment, the control unit 80 may divide the breast into the uncompressed region M0 and the compressed region M1 by using a sensing value of a pressure sensor installed on an upper surface of the detection unit 60 or the tray 62 positioned below the breast 1 or a lower surface of the compressing unit 50 positioned above the breast 1.

FIG. 6 is a view illustrating a state in which a pressure sensor unit is further provided in the mammography apparatus according to the exemplary embodiment of the present invention and a breast is positioned between the compressing unit and the detection unit.

FIG. 6 illustrates a state in which a pressure sensor unit 64 is provided on the upper surface of the tray 62. However, the pressure sensor unit 64 may be provided on the lower surface of the compressing unit 50. In a case in which a separate tray 62 is not provided, the pressure sensor unit 64 may be provided on an upper surface of the detection unit 60 and come into contact with the breast 1. The pressure sensor unit 64 may include a plurality of pressure sensors disposed in the form of an array. The positions of the plurality of pressure sensors disposed in the form of an array may match pixel information of the detection unit 60.

The control unit 80 may designate a region, in which a sensing value is less than a reference value, as the uncompressed region M0 on the basis of sensing information of the pressure sensor unit 64.

As described above, the control unit 80 may set the imaging condition for the main imaging on the basis of image information in the compressed region M1 except for the uncompressed region M0 in the image acquired by the preliminary imaging of the breast 1, and then allow the main imaging to be performed in accordance with the set imaging condition.

The control unit 80 may set the imaging condition for the main imaging by extracting an additional region of interest (ROI) in respect to the breast 1 as described below. This will be further described with reference to the mammography method according to the present invention.

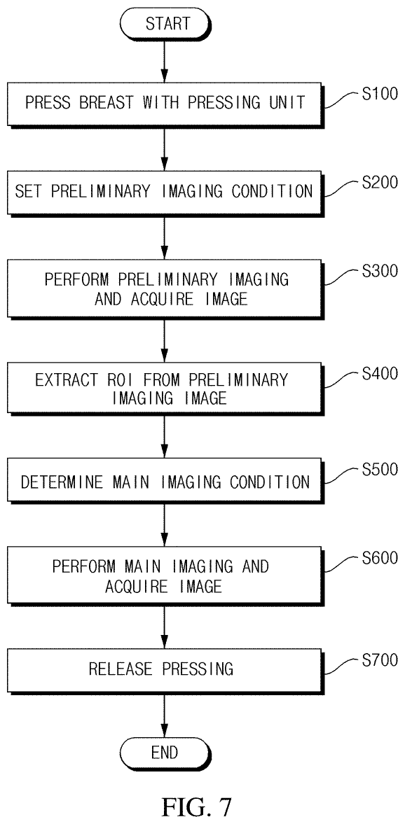

FIG. 7 is a flowchart illustrating the mammography method according to the exemplary embodiment of the present invention.

First, the compressing unit 50 compresses the breast 1 in the state in which the breast 1 is positioned on the detection unit 60 (S100).

Next, a preliminary imaging condition for the preliminary imaging of the breast 1 is determined (S200). The preliminary imaging condition may be the intensity, irradiation time, or dose of radiation from the irradiation unit 40. The preliminary imaging condition may be set by an operator or determined by the control unit 80 in accordance with a test purpose, imaging direction (such as CC or MLO) of the breast, or the thickness t of the compressed breast 1.

The preliminary imaging is performed on the breast 1 and the image is acquired on the basis of the determined preliminary imaging condition (S300). The image acquired by the detection unit 60 may be transmitted to the control unit 80.

The control unit 80 extracts the region of interest (ROI) from the preliminary imaging image (S400). In this case, the region of interest may be the compressed region M1 except for the uncompressed region M0 in the preliminary imaging image. In addition, the region of interest may be a region excluding a pectoralis region which is not the breast 1. In addition, the region of interest may be a region excluding an implant (implant) inserted into the breast 1. In addition, the region of interest may be a region including a mammary gland.

The control unit 80 determines the main imaging condition for the main imaging by using information on the region of interest extracted in step S400 (S500). In the exemplary embodiment, the main imaging condition may be set on the basis of the thickness t of the compressed breast 1 and/or density of the mammary gland.

The main imaging is performed in accordance with the main imaging condition to acquire a radiographic image of the breast (S600).

When the main imaging is ended, the pressing by the compressing unit 50 is released (S700).

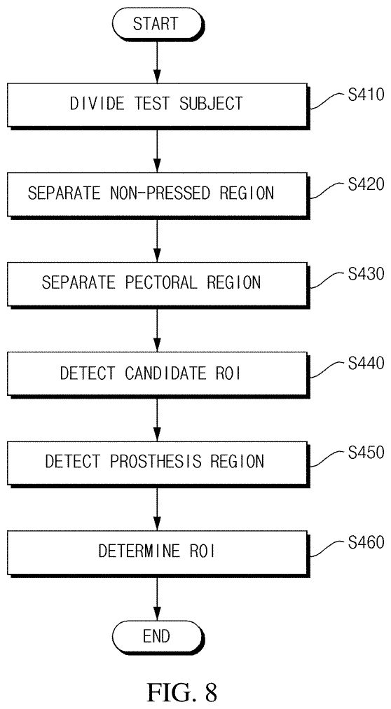

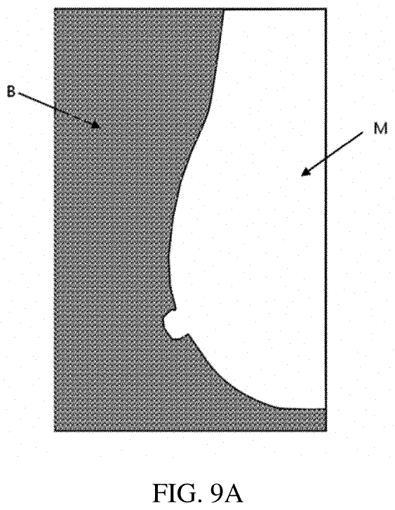

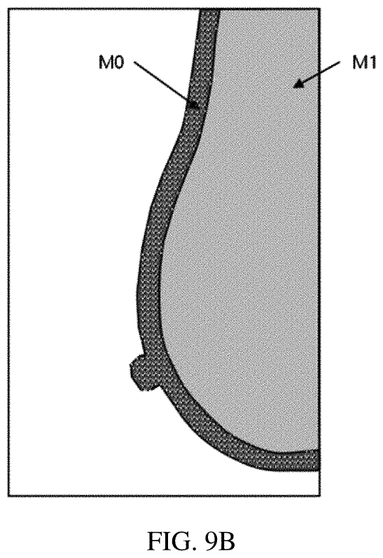

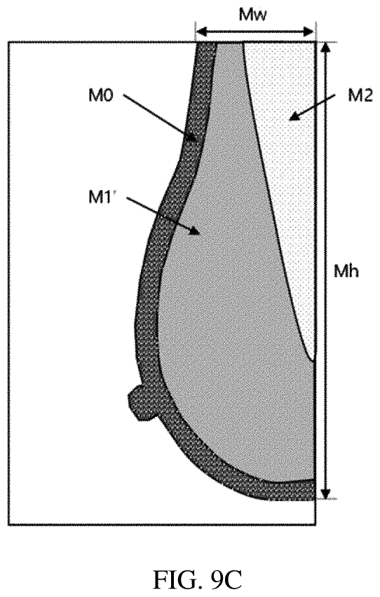

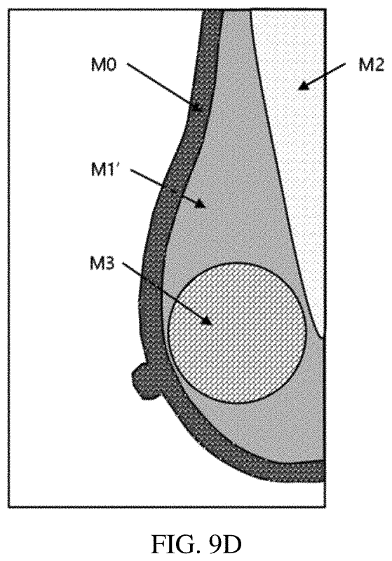

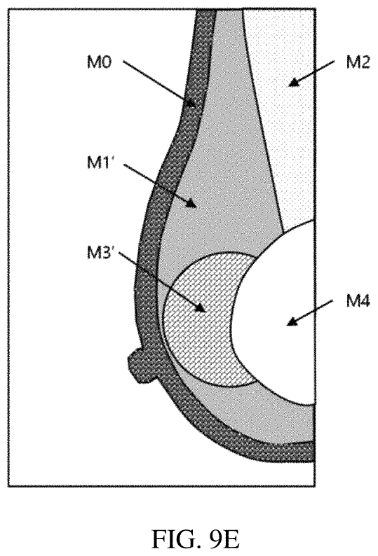

FIG. 8 is a flowchart illustrating a process of extracting the region of interest from the image acquired by preliminary imaging in the mammography method according to the exemplary embodiment of the present invention, and FIGS. 9A to 9E are views exemplarily illustrating a process of extracting the region of interest from the image acquired by preliminary imaging in the mammography method according to the exemplary embodiment of the present invention.

A process of extracting the region of interest from the image acquired by the preliminary imaging will be described below in detail.

The region of the image, which is acquired by the preliminary imaging and corresponds to the test subject, that is, the breast, is divided (S410). Referring to FIG. 9A, the image is divided into a background region B and a breast region M. This division may be performed by using the pixel values of the image, and a boundary between the breast and the background may be determined in consideration of deviations with the peripheral pixels or the amount of change in pixels.

Next, the breast region M is divided into the compressed region M1 and the uncompressed region M0. This is as described above, and the result thereof may be expressed as illustrated in FIG. 9B.

A pectoralis region M2 is separated from the compressed region M1 (S430). In particular, because the pectoralis region M2 may be included in the MLO image, it may be necessary to exclude the pectoralis region M2. In addition, because the pectoralis region M2 may not be included in the CC image, the step S430 may be included or omitted in accordance with the imaging direction.

Because the pectoralis region M2 has lower radiation transmittance than the region (M3 in FIG. 9D) including mammary gland, the pectoralis region M2 has low pixel values in the image. The pectoralis region M2 is separated from the image, and a compressed region Mr excluding the pectoralis region is extracted. As an example in which the pectoralis region M2 is separated, referring to FIG. 9C, the pectoralis region M2 is shown in an approximately right-angled triangular shape attached to one edge of a surface of a chest wall in the image, and the edge to which the chest wall is attached may be constant for each imaging (such as RMLO or LMLO). As illustrated in FIG. 9C, two lengths Mw and Mh in the breast region at the edge at which the pectoraliss are present are proportional to a width and a height of the pectoralis region shown in the image. Therefore, it is possible to approximately estimate the pectoralis region M2 in a right-angled triangular shape by multiplying Mw and Mh by a predetermined ratio, respectively. In addition, the pectoralis region is corrected on the basis of the pixel value in the estimated pectoralis region M2, and as a result, it is possible to more accurately determine and exclude the pectoralis region M2.

After the pectoralis region is excluded, the candidate region of interest is detected (S440). Referring to FIG. 9D, the candidate region of interest may be a region M3 including the mammary gland.

If an implant exists in the breast region, the implant region is detected (S450). Because the implant region does not transmit the radiation well, the implant region has smaller pixel values than other tissue in the image. Referring to FIG. 9E, an implant region M4 may be ascertained.

Finally, a final region of interest is determined (S460). In FIG. 9E, a final region of interest MY from which the implant region M4 is excluded may be ascertained.

Meanwhile, semantic segmentation may be used for at least one of the process of dividing the image acquired by the preliminary imaging into the background region B and the breast region M, the process of dividing the breast region M into the compressed region M1 and the uncompressed region M0, the process of separating the pectoralis region M2 from the compressed region M1, the process of detecting the region M3 including the mammary gland from the breast region M, and the process of detecting the implant region M4 from the breast region M.

The semantic segmentation means a process of assigning a class label to each pixel in the image acquired by the preliminary imaging. The semantic segmentation may be performed by using an artificial neural network such as a convolutional neural network (CNN) or an algorithm based on deep learning. In addition, the semantic segmentation may be performed by using techniques such as K-means clustering, Otsu's thresholding, or fuzzy clustering. In other words, the semantic segmentation may be performed in a supervised learning manner or an unsupervised learning manner.

According to the present invention, the final region of interest is extracted from the image acquired from the preliminary imaging, and the imaging condition for the main imaging is optimized, such that it is possible to obtain a clear radiographic result of the region of interest while reducing the radiation dose to be emitted to the breast.

The above description is simply given for illustratively describing the technical spirit of the present invention, and those skilled in the art to which the present invention pertains will appreciate that various modifications, changes, and substitutions are possible without departing from the essential characteristic of the present invention. Accordingly, the exemplary embodiments disclosed in the present invention and the accompanying drawings are intended not to limit but to describe the technical spirit of the present invention, and the scope of the technical spirit of the present invention is not limited by the exemplary embodiments and the accompanying drawings. The protective scope of the present invention should be construed based on the following claims, and all the technical spirit within the equivalent scope thereto should be construed as falling within the scope of the present invention.

As described above, the exemplary embodiments have been described and illustrated in the drawings and the specification. The exemplary embodiments were chosen and described in order to explain certain principles of the invention and their practical application, to thereby enable others skilled in the art to make and utilize various exemplary embodiments of the present invention, as well as various alternatives and modifications thereof. As is evident from the foregoing description, certain aspects of the present invention are not limited by the particular details of the examples illustrated herein, and it is therefore contemplated that other modifications and applications, or equivalents thereof, will occur to those skilled in the art. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A mammography method comprising:
a preliminary imaging step of performing imaging on a breast by emitting radiation in accordance with a first imaging condition;
a second imaging condition determination step of determining a second imaging condition in accordance with a first image acquired in the preliminary imaging step; and
a main imaging step of performing imaging on the breast by emitting the radiation on the basis of the second imaging condition,
wherein a uncompressed region in which the breast is not compressed by a compressing unit is excluded when determining the second imaging condition.

2. The mammography method of claim 1, wherein in the second imaging condition determination step, the uncompressed region is determined on the basis of a breast thickness t by which the breast is compressed by the compressing unit.

3. The mammography method of claim 2, wherein a length of the uncompressed region from a tip of the breast is smaller than the breast thickness t.

4. The mammography method of claim 2, wherein the uncompressed region is determined by modeling a front shape of the breast as any one of a hemispheric shape, an elliptical shape, and a parabolic shape.

5. The mammography method of claim 1, wherein in the second imaging condition determination step, the uncompressed region is determined in consideration of a change in pixel value in the first image.

6. The mammography method of claim 1, wherein in the second imaging condition determination step, the uncompressed region is determined by semantic segmentation on the first image.

7. The mammography method of claim 1, wherein the uncompressed region is determined by using a sensing value of a pressure sensor unit provided on an upper surface of a detection unit or a tray for supporting the breast or a lower surface of the compressing unit for compressing the breast.

8. The mammography method of claim 1, wherein the second imaging condition determination step further comprises a step of excluding a pectoralis region or an implant region from the first image.

9. The mammography method of claim 1, wherein the second imaging condition determination step further comprises a step of setting a mammary gland region to a region of interest in the first image, and determining the second imaging condition in consideration of the mammary gland region and the breast thickness t.

10. A mammography apparatus comprising:
an irradiation unit configured to emit radiation to a breast in accordance with a first imaging condition or a second imaging condition;
a compressing unit configured to compress the breast;
a detection unit configured to detect the radiation emitted to the breast; and
a control unit configured to determine the second imaging condition on the basis of a first image of the breast acquired in accordance with the first imaging condition, and control the irradiation unit and the detection unit,
wherein the control unit excludes a uncompressed region in which the breast is not compressed, when determining the second imaging condition.

11. The mammography apparatus of claim 10, wherein the control unit determines the uncompressed region on the basis of a breast thickness t by which the breast is compressed by the compressing unit.

12. The mammography apparatus of claim 11, wherein the control unit sets the uncompressed region to a distance from a tip of the breast, and the distance is smaller than the breast thickness t.

13. The mammography apparatus of claim 11, wherein the control unit determines the uncompressed region by modeling a front shape of the breast as any one of a hemispheric shape, an elliptical shape, and a parabolic shape.

14. The mammography apparatus of claim 10, wherein the control unit determines the uncompressed region in consideration of a change in pixel value in the first image.

15. The mammography apparatus of claim 10, wherein the control unit determines the uncompressed region by semantic segmentation on the first image.

16. The mammography apparatus of claim 10, wherein the breast is supported on an upper surface of the detection unit or a tray, a pressure sensor unit is provided on the upper surface of the detection unit or the tray or a lower surface of the compressing unit, and the control unit determines the uncompressed region by using a sensing value of the pressure sensor unit.

17. The mammography apparatus of claim 10, wherein the control unit sets a region of interest based on a mammary gland region by excluding a pectoralis region or an implant region from the first image.
